SEQUENCE LISTING (9 sequences)

SEQ ID NO: 1 - DNA, 46 bp, Artificial Sequence
  Oligonucleotide primer (FIP)
cagccagccgcagcacgttcgctcataggagatatggtagagccgc

SEQ ID NO: 2 - DNA, 51 bp, Artificial Sequence
  Oligonucleotide primer (BIP)
gagagaatttgtaccacctcccaccgggcacatagcagtcctagggacagt

SEQ ID NO: 3 - DNA, 22 bp, Artificial Sequence
  Oligonucleotide primer (F3)
ggcttggctctgctaacacgtt

SEQ ID NO: 4 - DNA, 22 bp, Artificial Sequence
  Oligonucleotide primer (B3)
ggacgtttgtaatgtccgctcc

SEQ ID NO: 5 - DNA, 17 bp, Artificial Sequence
  Oligonucleotide primer (Loop F)
ctgcatacgacgtgtct

SEQ ID NO: 6 - DNA, 20 bp, Artificial Sequence
  Oligonucleotide primer (Loop B)
accatctatgactgtacgcc

SEQ ID NO: 7 - DNA, 18 bp, Artificial Sequence
  TS primer
aatccgtcgagcagagtt

SEQ ID NO: 8 - DNA, 30 bp, Artificial Sequence
  ACX primer
gcgcggcttacccttacccttaccctaacc

SEQ ID NO: 9 - DNA, 62 bp, Artificial Sequence
  TSR8
aatccgtcgagcagagttagggttagggttagggttagggttagggttagggttagggttag

(19) United States
(12) Patent Application Publication
(10) Pub. No.: US 2019/0119737 A1
(43) Pub. Date: Apr. 25, 2019

(54) ENHANCED FLUORESCENCE READOUT AND REDUCED INHIBITION FOR NUCLEIC ACID AMPLIFICATION TESTS

(71) Applicant: THE REGENTS OF THE UNIVERSITY OF CALIFORNIA, Oakland, CA (US)

(72) Inventors: Dino Di Carlo, Los Angeles, CA (US); Janay Kong, Los Angeles, CA (US); Aydogan Ozcan, Los Angeles, CA (US); Omai Garner, Culver City, CA (US)

(73) Assignee: THE REGENTS OF THE UNIVERSITY OF CALIFORNIA, Oakland, CA (US)

(21) Appl. No.: 16/301,687

(22) PCT Filed: May 16, 2017
(86) PCT No.: PCT/US2017/032922

§ 371 Date: Nov. 14, 2018

Related U.S. Application Data
(60) Provisional application No. 62/337,433, filed on May 17, 2016.

(51) Int. Cl. C12Q 1/6844; C09B 29/16; C09B 23/04; C09B 15/00; C09B 69/04

(52) U.S. Cl. CPC C12Q 1/6844; C09B 29/16; C09B 69/045; C09B 15/00; C09B 23/04

(57) ABSTRACT

A fluorescent dye and quencher mixture for reporting on nucleic acid amplification from a sample includes a fluorescent intercalating dye, a dye sequestering or quenching agent such as hydroxynapthol blue (HNB) or caffeine, and primers, dNTPs, and a nucleic acid polymerizing enzyme or fragment thereof. The presence of the dye in combination with the dye sequestering or quenching agent improves the overall dynamic range of the fluorescent signal as well as shortens the time needed for visualization or image capture of amplified nucleic acid. The fluorescent dye and quencher mixture also enables the detection of nucleic acids in samples having low copy numbers.

### RELATED APPLICATION

This Application claims priority to U.S. Provisional Patent Application No. 62/337,433 filed on May 17, 2016, which is hereby incorporated by reference in its entirety. Priority is claimed pursuant to 35 U.S.C. § 119 and any other applicable statute.

### STATEMENT REGARDING FEDERALLY SPONSORED RESEARCH AND DEVELOPMENT

This invention was made with Government support under 1332275 from the National Science Foundation. The Government has certain rights in the invention.

### TECHNICAL FIELD

The technical field generally relates to methods of detecting and quantifying nucleic acid amplification using fluorescent intercalating dyes. In particular, the technical field generally relates to improved fluorescent dye and quencher mixtures or cocktails that are used to improve performance during nucleic acid amplification.

### BACKGROUND

Real-time or end-point fluorescence monitoring of nucleic acid amplification often requires costly custom probes or fluorescent intercalating dyes, such as EvaGreen®, which can interfere with nucleic amplification, and delay the time until result. For example, loop-mediated isothermal amplification (LAMP) is an isothermal nucleic acid amplification process that is increasing being used for nucleic acid amplification procedures because thermal cycling is not needed. Fluorescence detection based on intercalating dyes allows the fluorescent signal to be visualized both in bulk solution and in digital nanoliter volumes following isothermal amplification which is suitable for point-of-care devices. Details regarding the LAMP process may be found in Nagamine et al., Accelerated reaction by loop-mediated isothermal amplification using loop primers, Molecular and Cellular Probes, 16, 223-229 (2002), which is incorporated by reference as if set forth fully herein. Isothermal systems such as LAMP are able to eliminate the complexities of thermocycling, but have issues with signal generation above background levels. Current digital LAMP protocols measure fluorescence using intercalating dyes such as EvaGreen® but suffer from a delay in readout because intercalating dyes are known to interfere with nucleic amplification. This means that such dyes are added at the end of the amplification process if possible or in dilute quantities which impedes real-time measures of nucleic acids during the amplification process because of the reduced signal that is generated for dilute concentrations.

### SUMMARY

In one embodiment, a fluorescent dye and quencher mixture is used to report on nucleic acid amplification and achieves fluorescent signal generation that is an order of magnitude higher than previous techniques. Further, the mixture can be introduced during the beginning or prior to the amplification process without delaying amplification time. This fluorescent dye and quencher mixture has been applied to achieve highly sensitive loop-mediated isothermal amplification (LAMP). Improvements are also seen in other nucleic acid amplification methods such as polymerase chain reaction (PCR). In one particular embodiment, by using a conjugated dye, hydroxynapthol blue (HNB), to interact and sequester the fluorescent intercalating dye (e.g., EvaGreen®, SYBR® Green, or acridine orange) prior to oligonucleotide generation, the overall fluorescence fold change is improved and the time needed for fluorescent visualization of amplified nucleic acid is shortened.

As compared to current LAMP amplification systems which measure fluorescence using intercalating dyes such as EvaGreen®, SYBR® Green, or SYTO® dyes alone, the current mixture of fluorescent intercalating dye and the sequestering or quencher agent (e.g., HNB) exhibits a much higher overall fluorescence fold change over the unamplified background compared to current systems that use the intercalating dye alone. For the condition where EvaGreen® is used alone, fluorescence signal above background can be measured after 60-80 minutes, and the maximum fluorescent intensity is approximately 3-4 fold above background. In contrast, when HNB is included in the reaction mixture, the fluorescence signal can be measured much earlier; after 30-50 minutes, and the maximum fluorescent intensity is over 20-50 fold above background. Moreover, adding HNB to LAMP reactions with EvaGreen® stabilizes the fluorescent signal with respect to changes in temperature. Results using EvaGreen® and HNB in a digital LAMP readout system show that λ DNA concentrations of 57 copies/μl or lower can be distinguished above background.

This fluorescent intercalating dye and sequestering agent or quencher mixture allows for nucleic acid amplification to be measured in real-time (and at higher concentrations of dye). At the same time, another benefit with the mixture is that the time required to detect a “positive” result is significantly reduced. Similarly, the mixture lowers the limit-of-detection (LOD) when used in connection with digital readout systems. The temperature stability that HNB provides allows for this assay to be conducted in a point-of-care setting, and the universality of these dyes allow for ease of integration with any nucleic acid amplification techniques without the cost of custom fluorescent probes.

In one particular embodiment, a fluorescent dye and quencher mixture for reporting on nucleic acid amplification from a sample includes a fluorescent intercalating dye, hydroxynapthol blue (HNB), primers, dNTPs, and a nucleic acid polymerizing enzyme or fragment thereof. In one particular example, the amplification of the nucleic acid is done using LAMP amplification and the mixture includes LAMP primers, dNTPs, LAMP reaction buffer, and DNA polymerase or a fragment thereof. The fluorescent intercalating dye may include a dimeric fluorescent dye having an emission peak at around 530 nm (e.g., EvaGreen®), a cyanine dye having an emission peak at around 520 nm (e.g., SYBR® Green), or acridine orange.

In another embodiment, a fluorescent dye and quencher mixture for reporting on nucleic acid amplification from a sample includes a fluorescent intercalating dye, caffeine, primers, dNTPs, and a nucleic acid polymerizing enzyme or fragment thereof. The caffeine should preferably be at a relatively high concentration, for example, greater than or equal to 50 mM.

In another embodiment, a method of improving the fluorescent reporting of a nucleic acid amplification process that uses a fluorescent intercalating dye includes: providing a sample containing a nucleic acid sequence to be amplified and adding a mixture containing the fluorescent intercalating dye, hydroxynapthol blue (HNB), dNTPs, primers, and a nucleic acid polymerizing enzyme or fragment thereof.

In still another embodiment, a method of using the mixtures disclosed herein includes forming a plurality of small volumes from the mixture; imaging the plurality of small volumes; and identifying a subset of the plurality of small volumes that emit a positive fluorescent signal. The fluorescent signal of the small volumes may be read using an imager or reader device that reads the intensity levels of the individual small volumes. The positive fluorescent signal may be determined by a fluorescent signal that is at or above a pre-defined fluorescent intensity level. In one embodiment, the number of small volumes from the plurality that emit the positive fluorescent signal are counted or determined. Based on the number of positive small volumes, this count may be used to calculate, establish, or infer the concentration of nucleic acid in the sample. The small volumes may include droplets, emulsions, or microwells.

In another embodiment, a fluorescent dye and quencher mixture for reporting on nucleic acid concentration from a sample containing deoxyribonucleic acid (DNA) includes, in addition to the sample, a fluorescent intercalating dye and hydroxynapthol blue (HNB). In this embodiment, there is not amplification of DNA. Instead, the mixture is used to report out the amount or concentration of nucleic acid in the sample.

### BRIEF DESCRIPTION OF THE DRAWINGS

FIG. 1 schematically illustrates one embodiment of a fluorescent dye and quencher mixture.

FIG. 2 illustrates a plurality of sample holders or wells contained in a plate. The plate is read using a reader/imager device. In one embodiment, the reader/imager device may output a digital readout based on positive or negative results of the each sample holder in the plate.

FIGS. 3A-3D illustrate real-time fluorescence monitoring of nucleic acid amplification with EvaGreen® without HNB for varying concentrations of λ DNA using loop-mediated isothermal amplification (LAMP). FIG. 3A is 1× EvaGreen®. FIG. 3B is 0.5× EvaGreen®. FIG. 3C is 0.2× EvaGreen®. FIG. 3D is 0.1× EvaGreen®.

FIGS. 4A-4D illustrate real-time fluorescence monitoring of nucleic acid amplification with EvaGreen® combined with HNB for varying concentrations of λ DNA using loop-mediated isothermal amplification (LAMP). FIG. 4A is 1× EvaGreen® plus HNB. FIG. 4B is 0.5× EvaGreen® plus HNB. FIG. 4C is 0.2× EvaGreen® plus HNB. FIG. 4D is 0.1× EvaGreen® plus HNB.

FIGS. 5A and 5B illustrate endpoint fluorescent measurements of EvaGreen® (EvaGreen® only—FIG. 5A) vs. EvaGreen® with HNB (FIG. 5B) compared with the initial fluorescent measurements for varying concentrations of λ DNA using loop-mediated isothermal amplification (LAMP) at two temperatures (warm and room temperature).

FIGS. 6A and 6B illustrate endpoint fluorescent measurements of a mixture (1× (1.25 μM) or 20× dilution of original stock) containing EvaGreen® and HNB compared with the initial fluorescent measurements for two different endpoint times (50 minutes—FIG. 6A) and 60 minutes (FIG. 6B).

FIGS. 7A-7D illustrate real-time fluorescence monitoring of nucleic acid amplification with varying amounts of EvaGreen® combined with HNB for varying concentrations of λ DNA using loop-mediated isothermal amplification (LAMP). FIG. 7A is 5× EvaGreen® plus HNB. FIG. 7B is 4× EvaGreen® plus HNB. FIG. 7C is 2× EvaGreen® plus HNB. FIG. 7D is 1× EvaGreen® plus HNB.

FIG. 8A illustrates real-time fluorescence readings of LAMP using SYBR® green alone with varying amounts of λ DNA.

FIG. 8B illustrates real-time fluorescence readings of LAMP using SYBR® green in combination with 120 μM HNB with varying amounts of λ DNA.

FIGS. 9A-9D illustrate real-time fluorescence of LAMP using acridine orange alone (FIGS. 9A and 9B) and acridine orange in combination with HNB (FIGS. 9C and 9D) with varying amounts of λ DNA. The experiments of FIGS. 9A and 9C used 6.6 μM acridine orange. The experiments of FIGS. 9B and 9D used 13.3 μM acridine orange.

FIG. 10 illustrates a graph illustrating the fluorescent intensity plot of each fractionated volume contained in the microwells (total 1,936) of a compressed microfluidic device as a function of λ DNA copy number.

FIG. 11 includes a series of graphs illustrating the real-time quantitative PCR (qPCR) measurements of DNA amplification with EvaGreen® Master Mix with varying amounts of HNB added (0 HNB, 7.5 μM HNB, 15 μM HNB, 30 μM HNB). Rn is the EvaGreen® fluorescence signal without normalization to a reference dye.

FIG. 12 schematically illustrates a proposed mechanism of interaction between intercalating dyes and sequestration/quenching agents.

FIG. 13 illustrates a graph of fluorescent measurements taken over two temperature cycles ranging between 29° C. and 65° C. for EvaGreen® and EvaGreen® with HNB in deionized (DI) water. Improved temperature stability is seen in the sample containing HNB.

FIGS. 14A, 14B, and 14C illustrate, respectively, the fluorescence emission spectra (both in the presence of λ DNA and zero DNA) of acridine orange (FIG. 14A), SYBR® Green (FIG. 14B), and EvaGreen® (FIG. 14C) along with their corresponding molecular structures (presented below each respective spectral graph).

FIG. 15A illustrates the absorbance spectra for 120 μM HNB, 2.5 μM EvaGreen®, and both 2.5 μM EvaGreen® and 120 μM HNB with and without λ DNA.

FIG. 15B illustrates emission spectra (scale shown linearly) for the same samples and mixture of FIG. 14A.

FIG. 15C illustrates emission spectra (scale shown logarithmically) for the same samples and mixture of FIG. 14A.

FIG. 16A illustrates the absorption spectra for 13.3 μM acridine orange both with and without DNA. Also illustrated is the absorption spectra for 12 μM HNB (with and without DNA) and the absorption spectra of HNB and acridine orange (with and without DNA).

FIG. 16B illustrates a linear plot of the emission spectra for 13.3 μM acridine orange with 12 μM of HNB with and without DNA.

FIG. 16C illustrates a logarithmic plot of the emission spectra for 13.3 μM acridine orange with 12 μM HNB with and without DNA.

FIG. 17A illustrates the absorbance spectra for 1× SYBR® Green (SG) with 12 μM, 120 μM, and 1.2 mM HNB with and without λ DNA.

FIG. 17B illustrates the emission spectra for 1× SYBR® Green with 12 μM, 120 μM, and 1.2 mM HNB with and without λ DNA (plotted linearly).

FIG. 17C illustrates the emission spectra for 1× SYBR® Green with 12 μM, 120 μM, and 1.2 mM HNB with and without λ DNA (plotted logarithmically).

FIG. 18A illustrates real-time fluorescence measurements of λ DNA amplification with loop-mediated DNA amplification (LAMP) with 1.25 μM EvaGreen for different copy numbers of λ DNA.

FIG. 18B illustrates real-time fluorescence measurements of λ DNA amplification with loop-mediated DNA amplification (LAMP) with 1.25 μM EvaGreen and 5 mM caffeine for different copy numbers of λ DNA.

FIG. 18C illustrates real-time fluorescence measurements of λ DNA amplification with loop-mediated DNA amplification (LAMP) with 1.25 μM EvaGreen and 50 mM caffeine for different copy numbers of λ DNA. All error bars indicate s.d.

### DETAILED DESCRIPTION OF THE ILLUSTRATED EMBODIMENTS

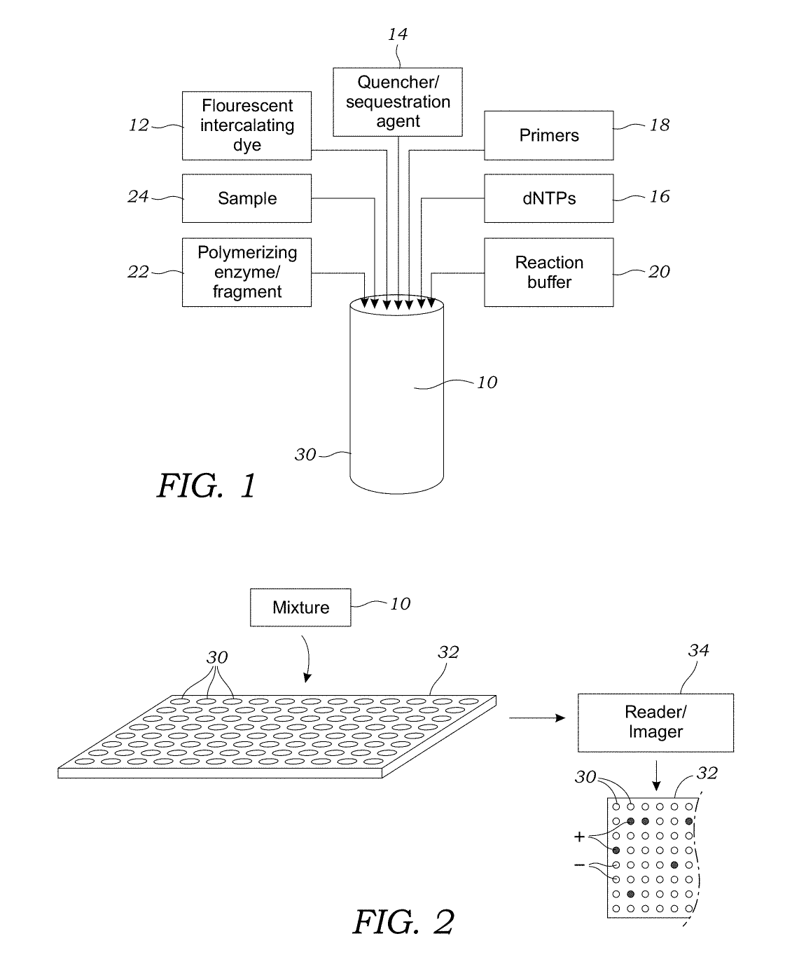

FIG. 1 schematically illustrates one embodiment of a fluorescent dye and quencher mixture 10. The inventors have unexpectedly and surprisingly discovered that the mixture 10 that combines a fluorescent intercalating dye 12, a quencher or sequestration agent 14, deoxynucleotide triphosphates (dNTPs) 16, primers 18, optional reaction buffer 20, a polymerizing enzyme or fragment thereof 22, along with a sample 24 containing a nucleic acid (e.g., deoxyribonucleic acid (DNA)) therein provides a significant increase in fluorescent signal change in a shorter period of time compared to current fluorescent reporting methods. The sample 24 contains, for example, a single or double stranded DNA sequence. This sequence may be known or unknown and is amplified using the mixture with fluorescent reporting via the fluorescent intercalating dye 12.

The quencher or sequestration agent 14 is a molecule that preferably has an affinity for the fluorescent intercalating dye 12 and/or is able to absorb the emitting fluorescent light from the intercalating dye 12. Stated differently, for the quencher or sequestration agent 14 there should be a degree of overlap between the absorption spectrum for the quencher or sequestration agent 14 and the fluorescence emission spectrum of the intercalating dye 12. In one particular example described herein, the quencher or sequestration agent 14 is hydroxynapthol blue (HNB). HNB is a commercially available azo dye having the empirical formula C₂₀H₁₁N₂Na₃O₁₁S₃. As explained herein, HNB significantly expands the dynamic range of the fluorescent signal that is generated during the nucleic acid amplification process. In another particular example, the quencher or sequestration agent 14 is caffeine.

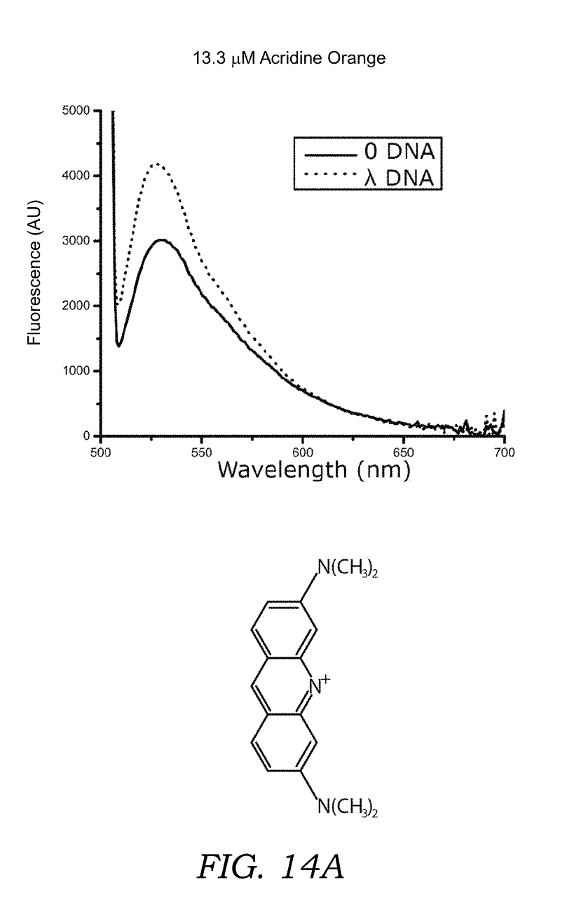

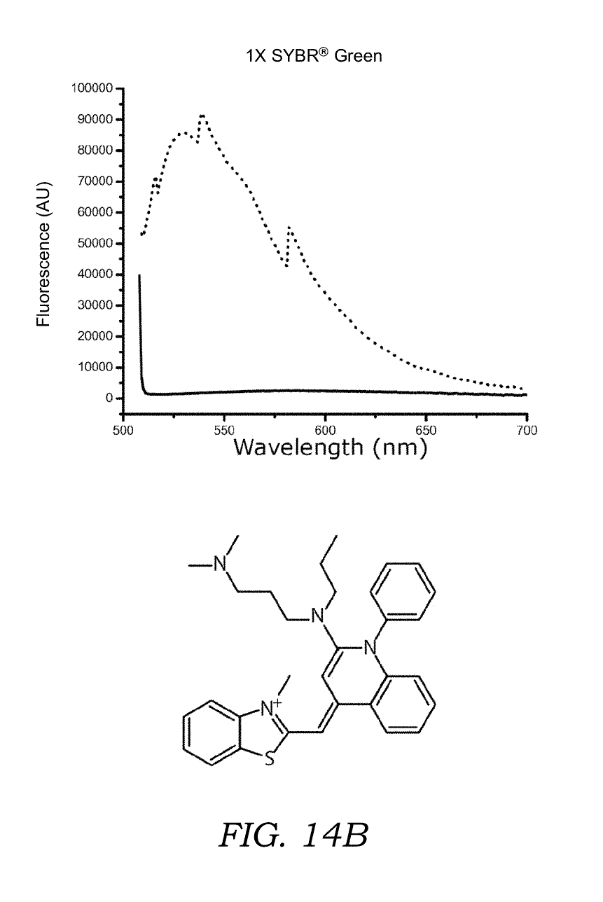

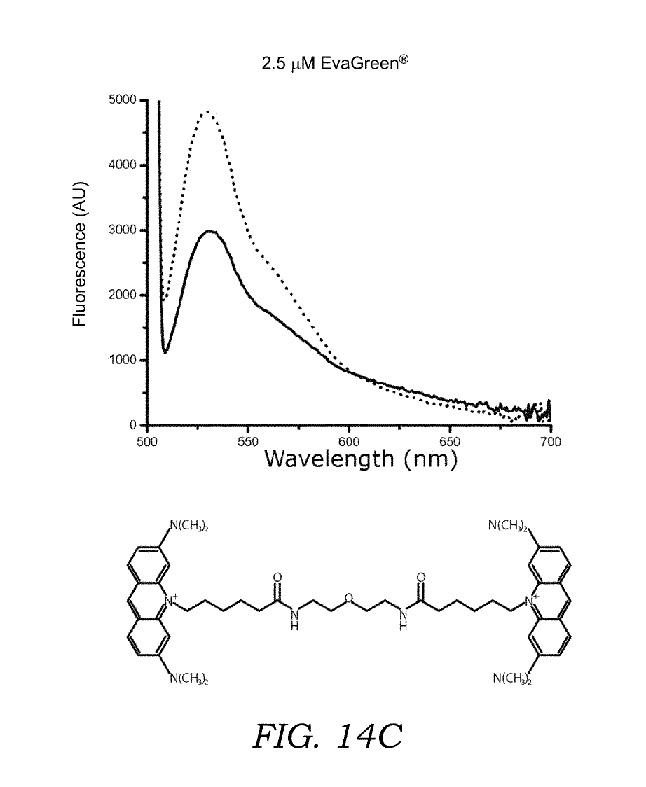

The fluorescent intercalating dye 12, as noted above, include those fluorescent intercalating dyes 12 that emit fluorescent light at a wavelength or wavelength range that overlaps with the absorption spectra of the quencher or sequestration agent 14. An example of a fluorescent intercalating dye 12 includes dimeric fluorescent dyes that bind to or have an affinity with nucleic acids and have an emission peak at around 530 nm. A commercial dye such as EvaGreen® available from Biotium, Inc. of Hayward, Calif. (e.g., Catalog #31000-T, 31000) is one example of such a dimeric fluorescent dye. Additional details regarding EvaGreen® may be found in U.S. Pat. Nos. 7,803,943 and 7,776,567, which are incorporated by reference herein. Another example of a fluorescent intercalating dye 12 that can be used with the mixture 10 are cyanine dyes having an emission peak at around 520 nm. A commercial example of such a dye includes SYBR® Green available from Thermo Fisher Scientific, Waltham, Mass. (Catalog #S7563). Another example of a fluorescent intercalating dye 12 that can be used with the mixture 10 includes acridine orange. FIGS. 14A, 14B, and 14C illustrate, respectively, the fluorescence emission spectra (both in the presence of λ DNA and zero DNA) of acridine orange, SYBR® Green, and EvaGreen® along with their corresponding molecular structures (presented below each spectral graph).

As seen in FIG. 1, the mixture 10 contains primers 18 which are unique to the amplification process that is used to amplify the nucleic acid in the sample 24. For example, the methods described herein can be used in connection with the LAMP amplification process, the PCR amplification process, as well as alternative amplification schemes such as NASBA (nucleic acid sequence based amplification), RCA (rolling circle amplification), MDA (multiple displacement amplification), Immuno-PCR, etc. The mixture 10 may also include an optional reaction buffer 20 that is used for the amplification process. Finally, the mixture 10 includes a polymerizing enzyme or enzyme fragment 22. This may include, for example, DNA polymerase or fragments thereof. Additional enzymes such as ligase or helicase may be needed with the mixture for the amplification of nucleic acid depending on the amplification process that is utilized.

With reference to FIGS. 1 and 2, the mixture is contained in a sample holder 30 which may take any number of forms. The sample holder 30 may include a cuvette, vial, well, microwell, or the like. In one particular embodiment as illustrated in FIG. 2, a plurality of sample holders 30 are provided in a substrate, plate 32 or the like in an array such as a 96 well plate that is commonly used. The various sample holders 30 in the plate may contain, for example, different fractions of the same sample or each sample holder 30 may contain different samples. As seen in FIG. 2, the plate 32 containing the sample holders 30 is placed in a reader/imaging device 34 whereby the array of sample holders 30 in the plate 32 are irradiated with excitation light (e.g., blue colored light in the case of the intercalating dyes 12 disclosed herein) and the array of sample holders 30 is imaged to capture fluorescent light that may be emitted from each sample holder 30 in response to nucleic acid amplification.

In one embodiment, the reader/imaging device 34 analyzes the intensity of fluorescent light emitted from each of the sample holders 30 (e.g., microwells, wells) of the plate 32 or other sample holder 30 containing device. The intensity may be monitored in real-time so that the time course of the amplification process can similarly be monitored. Alternatively, intensity measurements may be made at an end point after a certain time has expired or a certain number of amplification cycles have completed. In one particular embodiment, the fluorescent intensity of each sample holder 30 may be compared against a threshold intensity value by the reader/imaging device 34 to characterize a particular sample holder 30 as either positive or negative. In this regard, the sample holders 30 function to provide a digital readout that identifies each sample holder 30 (or fractionated volume as discussed below) as positive or negative. The positive sample holders 30 (or fractionated volumes) are those that have measured intensity levels that are at or above a pre-determined or pre-set threshold. Negative sample holders 30 (or fractionated volumes) are those with measured intensity levels that are below this same threshold. FIG. 2 illustrates, positive (+) sample holders 30 and negative (−) sample holders 30. In one particular embodiment, the reader/imaging device 34 is able to count the total number of positive sample holders 30 and use this information to characterize the initial concentration or copy number of nucleic acid in the initial sample 24. For example, Poisson distribution of nucleic acid molecules within fractionated volumes that are contained in the sample holders 30 can be used to determine the initial concentration or copy number.

As an alternative to well-based sample holders 30 (e.g., microwells), the fractional volumes may also be formed in small droplets or emulsions. These small droplets or emulsions act as discrete sample holders 30 and can then be imaged and analyzed using the reader/imaging device 34. For example, these droplets or emulsions could be formed using known microfluidic device designs that generate pinched aqueous-based droplets using oil-based pinching flows. These droplets or emulsions may be collected downstream of their generation and then imaged using the reader/imaging device 34. In still another embodiment, fractionated volumes of sample can be located in individual microwell sample holders 30 that are created between a two-layer, compression based device. For example, microwells formed in a polydimethylsiloxane (PDMS) substrate can be compressed against an optically transparent flat substrate like a glass slide. An inner volume is formed between the two layers and, when brought together in a compression process, forms a plurality of discrete, fractionated volumes.

In one embodiment of the invention, the fluorescent intercalating dye and quencher/sequestration agent mixture is used for LAMP-based amplification of nucleic acid. In one embodiment of the invention, the mixture includes the sample (i.e., the sample that contains the nucleic acid or DNA that is to be amplified), an intercalating dimeric fluorescent dye having an emission peak at around 530 nm such as EvaGreen® (e.g., Catalog #31000-T, 31000 available from Biotium, Inc. of Hayward, Calif.). The mixture also includes hydroxynapthol blue (HNB) (e.g., 120 μM); and LAMP primers which includes FIP, BIP, F3, B3, Loop F, and Loop B as shown below in Table 1, dNTPs, LAMP reaction buffer, and DNA polymerase.

TABLE 1

| Primer | Sequence (5′−>3′) |
|---|---|
| FIP | SEQ ID NO: 1 |
| BIP | SEQ ID NO: 2 |
| F3 | SEQ ID NO: 3 |
| B3 | SEQ ID NO: 4 |
| Loop F | SEQ ID NO: 5 |
| Loop B | SEQ ID NO: 6 |

While EvaGreen® shows the best results it should be understood that other intercalating dyes can be used. Other examples include, for example, a cyanine-based fluorescent intercalating dye having an emission peak at around 520 nm (e.g., SYBR® Green) or acridine orange dyes.

Example #1: The following is an exemplary mixture in accordance with one embodiment. Note that in this example, the DNA that is to be amplified is λ DNA (Thermo Scientific, SD0011), which is a linear double-stranded lambda bacteriophage (cI857 Sam7) DNA, 48502 base pairs with a molecular weight of 31.5×10⁶ Da. isolated from a heat-inducible lysogenic E. coli W3110 strain. GeneBank/EMBL accession numbers J02459, M17233, M24325, V00636, X00906. The LAMP reaction buffer includes 20 mM Tris-HCl (pH 8.8), 10 mM KCl, 10 mM ammonium sulfate, 8 mM magnesium sulfate, 1 M Betaine, 0.1% Triton-X 100, and 1.6 mM dNTPs. The LAMP reaction was carried out in 100 μl volumes on a 96-well plate in triplicates. 10 μl of serially diluted λ DNA, 0.64 μM FIP and BIP, 0.08 μM F3 and B3, 0.16 μM Loop F and Loop B, 32 units Bst DNA polymerase large fragment (Catalog #M0275L from New England Biolabs, Inc., Ipswich, Mass.), 120 μM HNB, and varying amounts of EvaGreen® were used in the LAMP reaction buffer. The stock solution of EvaGreen® that was used was a 20× solution of 25 μM EvaGreen® that was diluted as illustrated herein. Two negative controls, 0 DNA and 0 DNA w/o polymerase were used. For experimental results measuring the fluorescent signal, a Biotek plate reader set at 65° C. for 2.5 hours was used. In experiments relating to this example, 120 μM HNB was used.

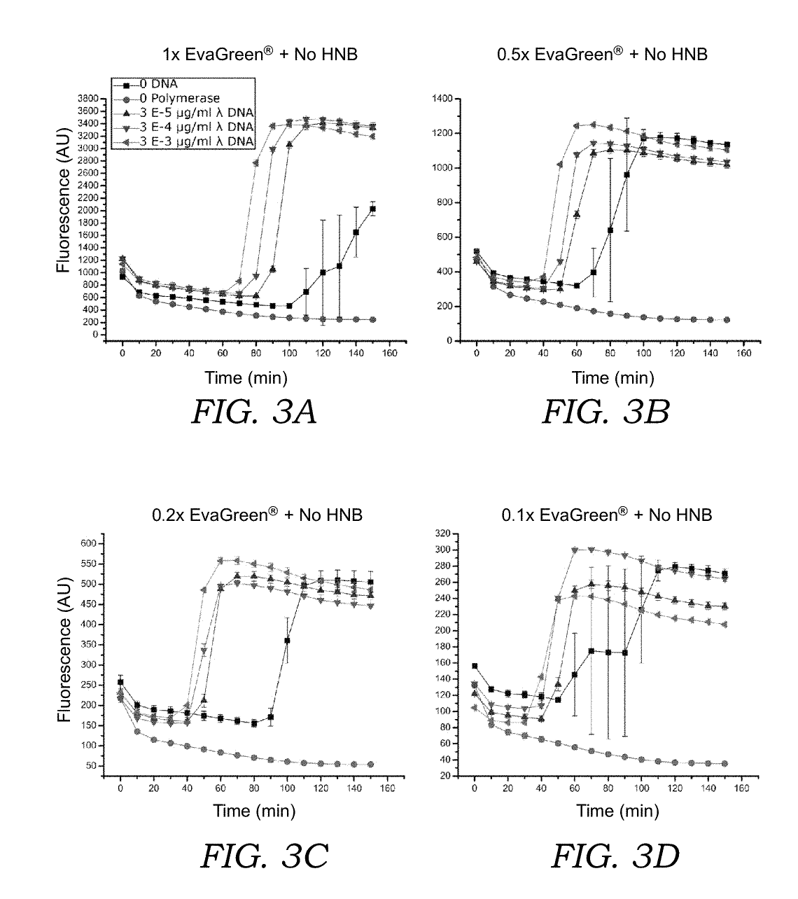

FIGS. 3A-3D illustrate the real-time fluorescence monitoring of nucleic acid amplification with EvaGreen® and without HNB for varying concentrations of λ DNA using loop-mediated isothermal amplification (LAMP). Note that the EvaGreen® complexes are unstable at high temperatures for LAMP and have a slow decay in fluorescence intensity over time. Additionally, higher concentrations of EvaGreen® are seen to interfere with amplification, increasing the time until signal increase for all concentrations of DNA (e.g., compare 1× EvaGreen® with other measurements). Without HNB the initial fluorescence intensity at time 0 increases with increasing EvaGreen® concentration, suggesting that a fraction of the dye is unquenched by solely EvaGreen® dimer interactions at 65° C., yielding high background fluorescence.

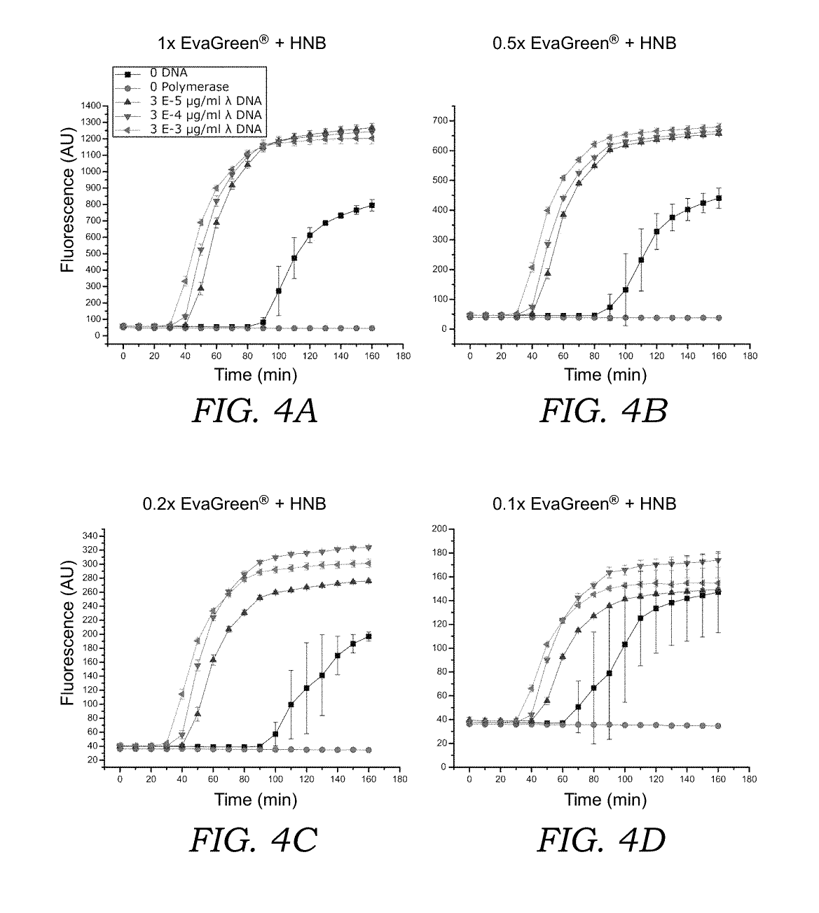

FIGS. 4A-4D illustrate real-time fluorescence monitoring of nucleic acid amplification with EvaGreen® combined with HNB for varying concentrations of λ DNA using loop-mediated isothermal amplification (LAMP). Note the overall increase in fluorescence fold changes as compared to baseline as well as the shorter start time for the initial increase in fluorescence compared to EvaGreen® alone (i.e., FIGS. 3A-3D), especially at higher EvaGreen® concentrations. Fluorescence intensity at time 0 is relatively independent of EvaGreen® concentration, suggesting interaction with HNB strongly quenches fluorescence. Note that the intensity does not fluctuate with time over the first 30 minutes suggesting HNB also stabilizes EvaGreen® at high temperature in a stable quenched form. Further note the improvement in the baseline stability (e.g., less drift over time) when the mixture having both EvaGreen® and HNB is used.

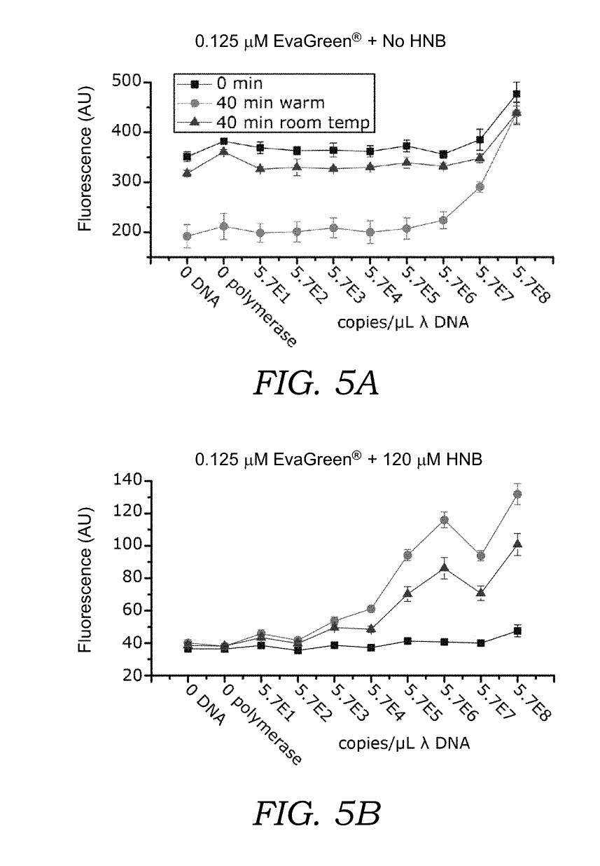

FIGS. 5A and 5B illustrate endpoint fluorescent measurements of EvaGreen® (only) vs. EvaGreen® with HNB compared with the initial fluorescent measurements. Endpoint measurements are taken immediately after the reaction takes place (time=0), while the plate is still warm (approx. 55-65° C.) (time=40 minutes), and after the plate cools to room temperature (time=40 minutes). Notice that for the EvaGreen® (only), the increasing trend in fluorescence with increasing amounts of DNA almost disappears after the plate cools to room temperature, whereas the addition of HNB not only stabilizes the fluorescence, but also improves the trend such that the limit of detection is significantly lower. The addition of HNB thus provides temperature stability and the assay can be conducted in point-of-care settings where temperature control cannot be readily employed or where temperatures vary.

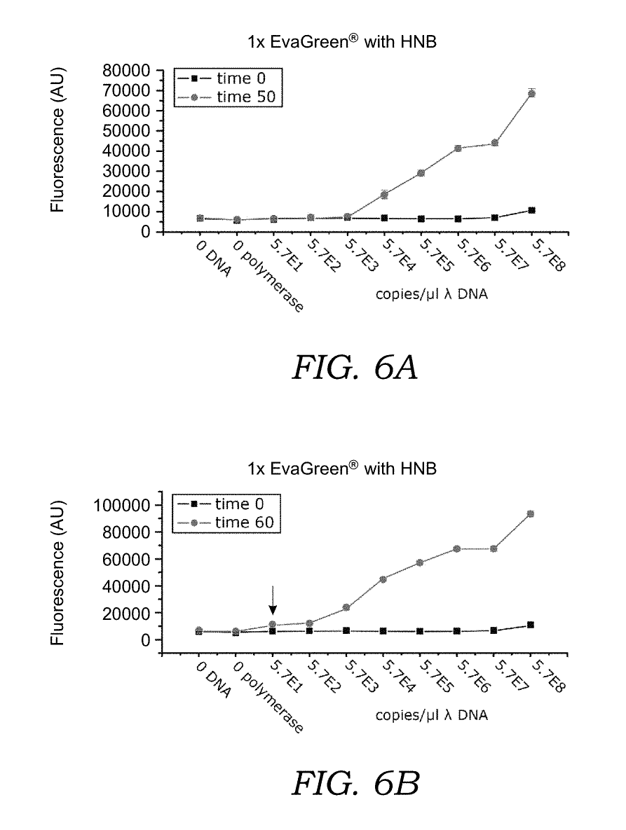

FIGS. 6A and 6B illustrate endpoint fluorescent measurements of a mixture (1× which corresponds to 20× dilution of original stock) containing EvaGreen® and HNB compared with the initial fluorescent measurements. FIG. 6A illustrates end point measurements taken at 50 minutes while FIG. 6B illustrates end point measurements taken at 60 minutes. Notice that for the longer elapsed time (FIG. 46), the limit of detection (LOD) for the assay decreases whereby the assay in FIG. 6B is able to discern or detect 57 copies/μl of λ DNA as seen by the arrow in FIG. 6B. An additional assay runtime of ten (10) extra minutes shows the LOD decreases by several orders of magnitude.

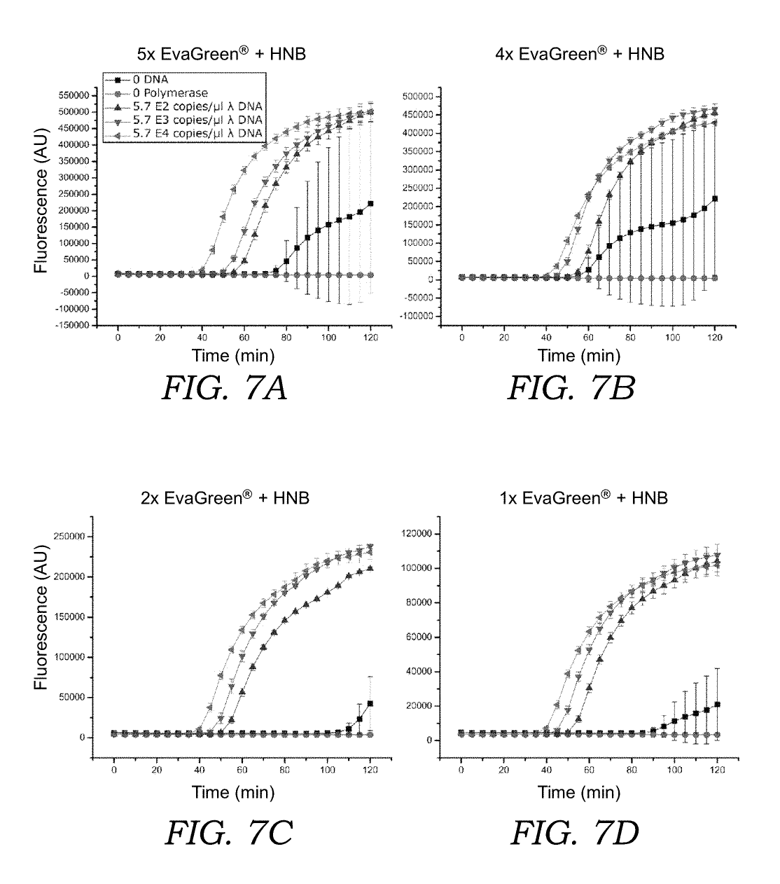

FIGS. 7A-7D illustrate real-time fluorescence monitoring of nucleic acid amplification with varying amounts of EvaGreen® combined with HNB for varying concentrations of λ DNA using loop-mediated isothermal amplification (LAMP). Note the overall fluorescent fold changes for the 4× and 5× experiments are approximately 70. For the 2× experiment the fluorescent fold change is approximately 50. For the 1× experiment the fluorescent fold change is approximately 20. Also, the start times for the initial, measurable increase in fluorescence do not change much with respect to the EvaGreen® concentration. Based on these findings, a 4× EvaGreen® solution (5 μM) may be the optimal concentration with this particular amplification process. It should be understood, however, that varying concentrations of EvaGreen® can be used with the mixture described herein.

Example #2: The following is an exemplary mixture according to another embodiment. The DNA that was amplified was λ DNA (Thermo Scientific, SD0011). The LAMP reaction buffer includes 20 mM Tris-HCl (pH 8.8), 10 mM KCl, 10 mM ammonium sulfate, 8 mM magnesium sulfate, 1 M Betaine, 0.1% Triton-X 100, and 1.6 mM dNTPs. The LAMP reaction was carried out in 100 μl volumes on a 96-well plate in triplicates. 10 μl of serially diluted λ DNA, 0.64 μM FIP and BIP, 0.08 μM F3 and B3, 0.16 μM Loop F and Loop B, 32 units Bst DNA polymerase large fragment (Catalog #M0275L from New England Biolabs, Inc., Ipswich, Mass.), 120 μM HNB, and a 1× dilution of SYBR® Green was used in the LAMP reaction buffer. The stock solution of SYBR® Green that was used was a 10,000× solution (Catalog #S7563 from Thermo Fisher Scientific, Waltham, Mass.) that was diluted 10,000 times as illustrated herein. Two negative controls, 0 DNA and 0 DNA without (w/o) polymerase were used. For experimental results measuring the fluorescent signal, a Biotek plate reader set at 65° C. for 2.5 hours was used. In all experiments in this example 120 μM HNB was used.

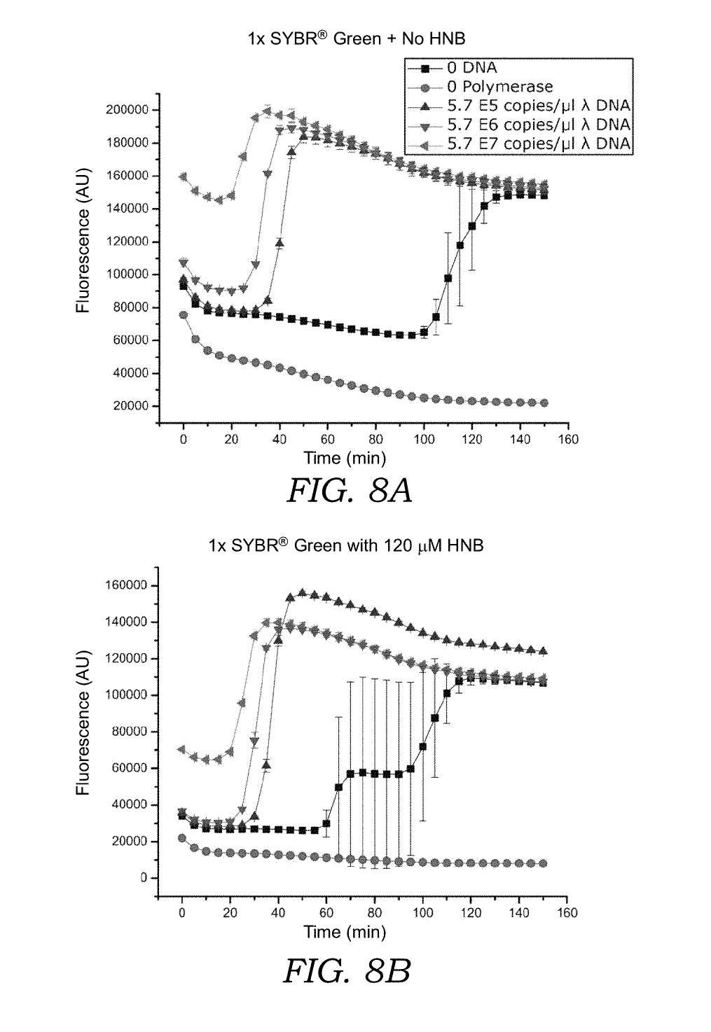

FIG. 8A illustrates real-time fluorescence readings of LAMP using SYBR® green alone while the real-time fluorescence readings of SYBR® green in combination with HNB are illustrated in FIG. 8B. The fluorescence fold change for SYBR® green alone is approximately 2×. The fluorescence fold change for SYBR® green when used in combination with HNB increases to approximately 3×. Additionally, the decrease in fluorescence at the initial time points is minimized when HNB is introduced, and the time for fluorescence visualization of the amplification is also slightly shortened with the presence of HNB. As seen in FIG. 8B, the baseline signal is more stable for the mixture that includes SYBR® green in combination with HNB.

Example #3: The following is an exemplary mixture according to another embodiment. The DNA that was amplified was λ DNA (Thermo Scientific, SD0011). The LAMP reaction buffer included 20 mM Tris-HCl (pH 8.8), 10 mM KCl, 10 mM ammonium sulfate, 8 mM magnesium sulfate, 1 M Betaine, 0.1% Triton-X 100, and 1.6 mM dNTPs. The LAMP reaction was carried out in 100 μl volumes on a 96-well plate in triplicates. 10 μl of serially diluted λ DNA, 0.64 μM FIP and BIP, 0.08 μM F3 and B3, 0.16 μM Loop F and Loop B, 32 units Bst DNA polymerase large fragment (Catalog #M0275L from New England Biolabs, Inc., Ipswich, Mass.), 120 μM HNB, and a two different concentrations of acridine orange (6.6 μM and 13.3 μM) was used in the LAMP reaction buffer. Acridine orange was purchased from Sigma-Aldrich, St. Louis, Mo. (Catalog #A9231—2% in water). Two negative controls, 0 DNA and 0 DNA w/o polymerase were used. For experimental results measuring the fluorescent signal, a Biotek plate reader set at 65° C. for 2.5 hours was used. In all experiments in this example that used HNB, 120 μM HNB was used.

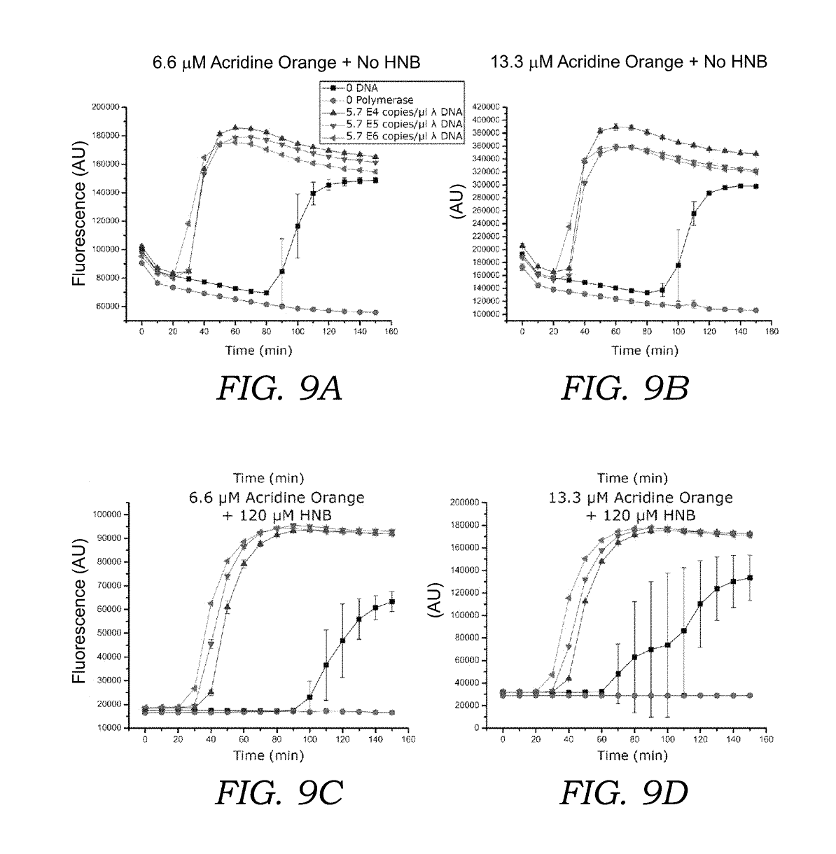

FIGS. 9A-9D illustrate real-time fluorescence of LAMP using acridine orange alone (FIGS. 9A and 9B) and acridine orange in combination with HNB (FIGS. 9C and 9D). The fluorescence fold change for both concentrations of acridine orange used without HNB is approximately 2×. The fluorescence fold change for both concentrations of acridine orange used when used with HNB increases to approximately 5×. Note: In the cases with HNB present, the initial fluorescence decrease seen in FIGS. 9A and 9B without HNB is no longer present. Better baseline stability is also seen in the mixtures that included HNB as seen in FIGS. 9C and 9D. Additionally, the fluorescence decreasing that appears at later time points, potentially due to thermal degradation of the intercalator dye, is not seen when HNB is present in solution.

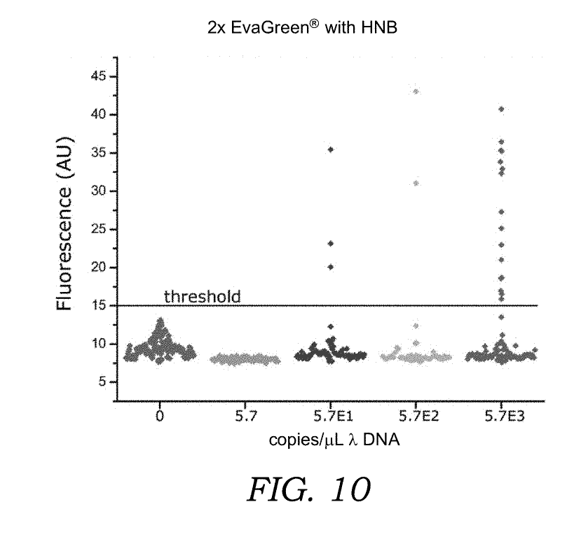

FIG. 10 illustrates a graph of the fluorescent intensity of each microwell as a function of λ DNA copy number for 2× EvaGreen® with HNB. Microwells were formed between an optically transparent flat substrate like a glass slide that was compressed against a polydimethylsiloxane (PDMS) substrate. The compression forms a plurality of discrete, fractionated volumes. In the embodiments used for the experimental results described herein the microwells were 200 μm in diameter and 65 μm in height. The entire device had 1,936 microwells located in a 1×1 cm² area. An arbitrary fluorescent intensity threshold level was set at 15 a.u. to demarcate positive microwells from negative microwells. Alternatively, thresholds could be set based on baseline levels or baseline levels plus a measure of variance of empty control wells or wells with reaction mixture without polymerase or other enzyme. As seen in FIG. 10, samples with 57 copies/μL or more can be identified as exhibiting positive signals (i.e., above threshold).

While the combination of the intercalating dye and HNB has largely been described in the context of the LAMP amplification process it should be understood that the intercalating dye and HNB can be used with other solutions that contain all the necessary components for nucleic acid amplification using alternative methods such as PCR (polymerase chain reaction), NASBA (nucleic acid sequence based amplification), RCA (rolling circle amplification), MDA (multiple displacement amplification), Immuno-PCR, etc.

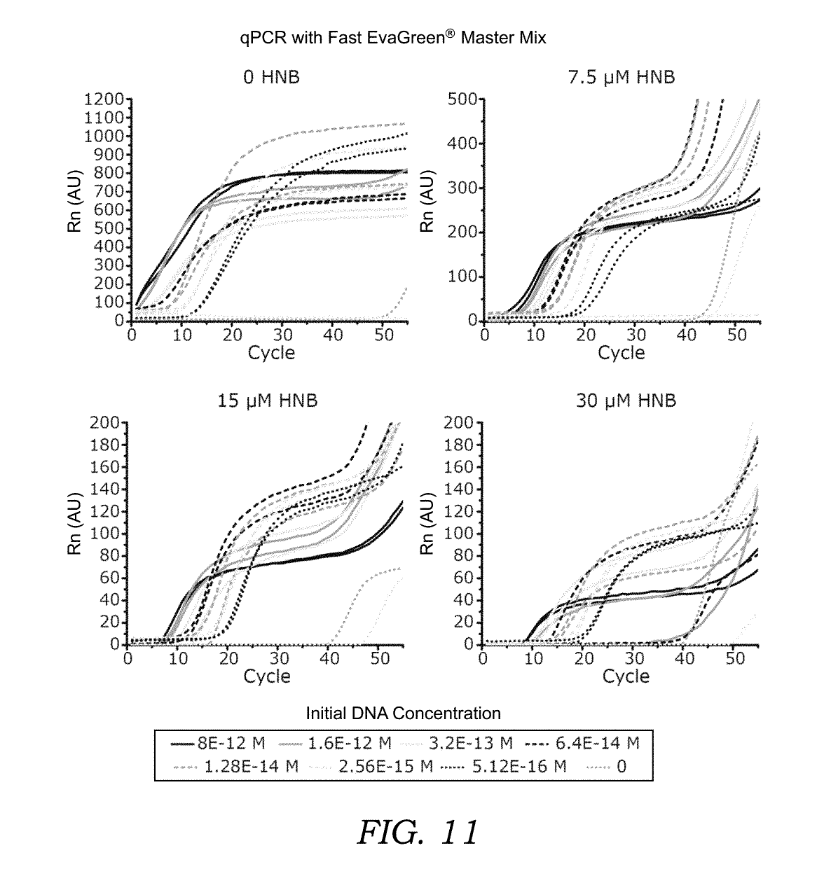

Example #4: Quantitative PCR (qPCR) was performed on Applied Biosystems 7500 Fast Real-time PCR instrument using the Biotium Fast EvaGreen® master mix according to the manufacturer's specifications. Briefly, 4 ng/ul of TS primer [SEQ ID NO: 7], 2 ng/μl of ACX primer [SEQ ID NO: 8] were added to the master mix with varying amounts of DNA, and ultrapure water. Each reaction was conducted in a qPCR plate in 20 μl volumes. The initial enzyme activation step was conducted at 95° C. for 2 min, and then cycled 55 times with 15 seconds at 95° C. and 60 seconds at 60° C. TSR8 DNA [SEQ ID NO: 9] was used as the starting material at varying concentrations. As shown in FIG. 11, the addition of HNB to the qPCR reaction mixture is shown to decrease the variation in Rn (the EvaGreen® fluorescence signal without normalization to a reference dye) seen in the earlier cycles. It also decreases the variation in the cycle threshold (the number of cycles to increase intensity above a threshold). For example for 7.5 μM of HNB, the cycle thresholds for repeat samples of the same concentration of DNA have a lower standard deviation. The baseline (initial) intensities are also more uniform and the shapes of the amplification curves overall are more repeatable with HNB compared to using the EvaGreen® intercalator without added HNB. Interestingly, there is a second increase in intensity at later cycle numbers which also appears to be dependent on the initial concentration of spiked TSR8 DNA.

The fluorescence emission that is generated following nucleic acid amplification is dependent on the fluorescence of the individual components of the assay as well as any complexes formed. Further investigation was performed to determine the emission spectra of each component and complex formed and examine when it is more favorable to form a complex between an intercalator and sequestration molecule that quenches fluorescence versus an intercalator and a DNA molecule, which would affect the overall fluorescence intensity before (low DNA concentrations) and following (high DNA concentrations) a nucleic acid amplification reaction.

Investigation showed that intercalating dyes such as EvaGreen®, SYBR® Green, and acridine orange have strong Förster resonance energy transfer (FRET) and/or quenching interactions, and these interactions lead to a decrease in the baseline fluorescence signal for solutions without DNA, measured at 535 nm, when added with a dye, such as HNB that has absorption near the emission maximum of these intercalating dyes. Additionally, when the absorbance and emission spectra for LAMP solutions with and without DNA are examined pre- and post-amplification, the relative affinities and fluorescence intensity of the several possible complexes between the intercalating dyes, the chemical additive, DNA, and subsequent complexes are elucidated. EvaGreen® and HNB interact in a manner that HNB sequesters the intercalating dye when there is only a small amount of DNA present.

However, after amplification, when there is a large accumulation of DNA, the EvaGreen® binding shifts from the HNB to DNA, where its quantum efficiency increases, generating an increase in fluorescence signal compared to a much lower background signal from the quenched EvaGreen®:HNB complex. Understanding the mechanics of these dye interactions allow for further development, optimization, and discoveries for the addition of an intercalating dye and sequestration and quenching molecule to a nucleic acid amplification assay. Besides HNB, other additives that bind with high affinity to EvaGreen® or other intercalators and also are suitable resonance energy transfer/quenching pairs could be used to improve signal to noise in these systems. Importantly, these additives should be soluble in aqueous solution and also not have an affinity or interact with polymerases or other enzymes used in nucleic acid amplification.

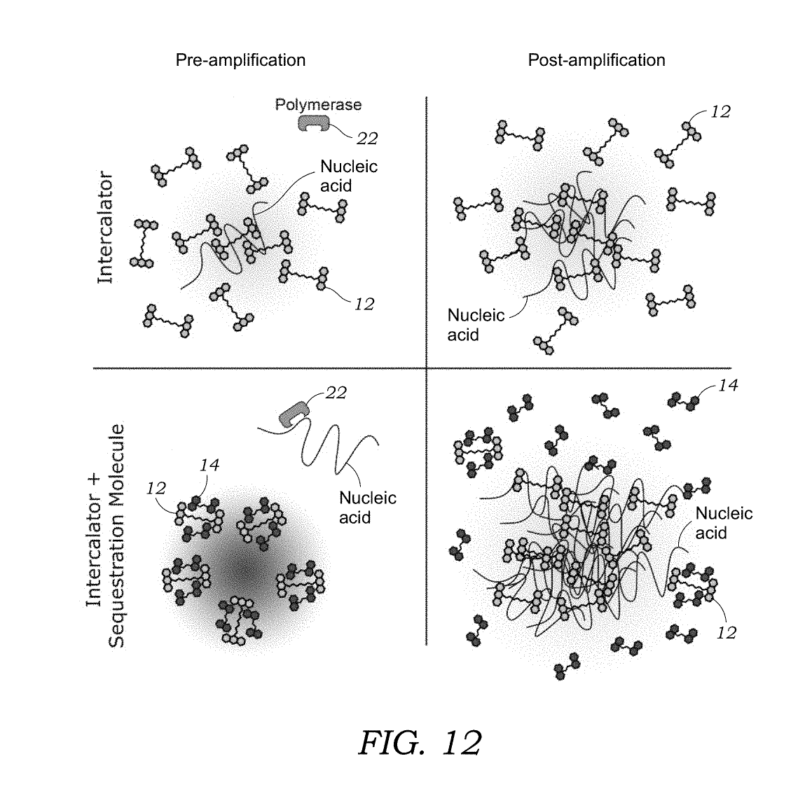

While not wishing to be bound to a particular theory, the proposed interaction between a fluorescent intercalating dye and the quenching/sequestration agent is illustrated in FIG. 12. Prior to amplification, if an intercalator dye molecule is present in solution (without a quenching/sequestration agent), the interaction with DNA will interfere with the amplification process. Conversely, if a sequestration molecule (e.g., HNB) is present at sufficient concentration, the molecule will sequester the intercalator dye molecule, forming a sequesterer:intercalator complex, and allowing amplification. The sequesterer:intercalator complex also preferably has increased stability to temperature and light exposure compared to the intercalator alone. The sequesterer preferably interacts via FRET or quenching with the intercalator to decrease the background fluorescence signal. Once amplification occurs, DNA concentration increases and the equilibrium then shifts such that intercalator:DNA complexes are more prevalent. Intercalator:DNA complexes have a high quantum efficiency for fluorescence allowing for a strong fluorescence signal to be measured.

Intercalator:DNA complexes which dissociate upon a temperature increase are also used in high resolution melting (HRM) curve analysis. EvaGreen® is one example of an intercalator that is widely used in HRM because it is a saturating dye that is known to fill the majority of intercalating sites (as opposed to SYBR® Green). Saturating sites is important to prevent dye “jumping” during melting curve analysis. The addition of an intercalator sequestering agent like HNB to HRM also can improve the accuracy and stability of HRM analysis. The temperature stability imparted by HNB can decrease the need for calibration/normalization of the curves, and the ability of HNB to quench released EvaGreen® upon increasing temperature reduces the background fluorescent signal of the unbound dye, leading improved peak-finding in the melting curve. The improved peak-finding could enable for more multiplexing and readout of different amplification reactions based on melting point analysis with higher definition.

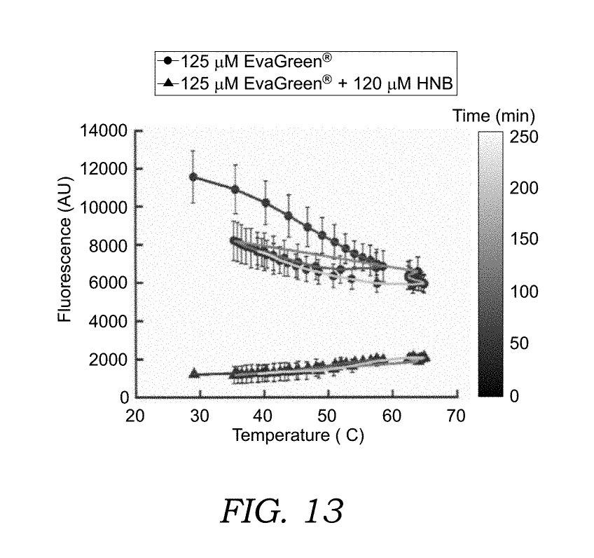

FIG. 13 illustrates how HNB interacts with an intercalating dye EvaGreen® to stabilize the fluorescent intensity with changes in temperature even without the presence of DNA in solution. Temperature stability is essential in assays developed for point-of-care or field use. In order to examine the interactions between EvaGreen® and HNB directly, the fluorescence intensity of a solution (1.25 μM) of EvaGreen® without DNA in deionized (DI) water was measured over two temperature cycles between ˜30-65° C. and compared with the same solution with added HNB (120 μM). FIG. 13 illustrates that with increasing temperature, the fluorescence decreases in the solution without HNB, whereas the fluorescence of the solution with HNB increases with temperature. The solution without HNB displays hysteresis, with the fluorescence dependent on the cycle number of the temperature cycle, not returning to the same intensity when returning to the same temperature in cycle number 2 at a later time compared to cycle number 1 at an earlier time. Additionally, the range of fluorescent intensities is much larger in solution without HNB (6000-11000 AU) versus (1800-2800 AU) in the solution with HNB. Overall, the temperature-induced changes in fluorescence are much greater when HNB is not present in solution with EvaGreen®. This large instability with temperature using EvaGreen® alone makes it more difficult to interpret changes in fluorescence as a result of DNA amplification from changes due to temperature fluctuation. The ability for the fluorescence to remain stable across a range of temperatures is especially important in point-of-care or low-resource settings.

The emission spectra for various intercalating dyes were examined to compare the signal in the presence of DNA at 535 nm compared to the background without DNA (See FIGS. 14A, 14B, 14C). Emission and absorbance spectra measurements were taken at room temperature on a Biotek Cytation 5 plate reader. The LAMP reaction was performed as described previously. For the LAMP assay measurements, readings were taking at time 0 and after 60 minutes incubation at 65° C. for reactions without DNA, without DNA and polymerase, and with 5.7E3 copies/μL λ DNA.

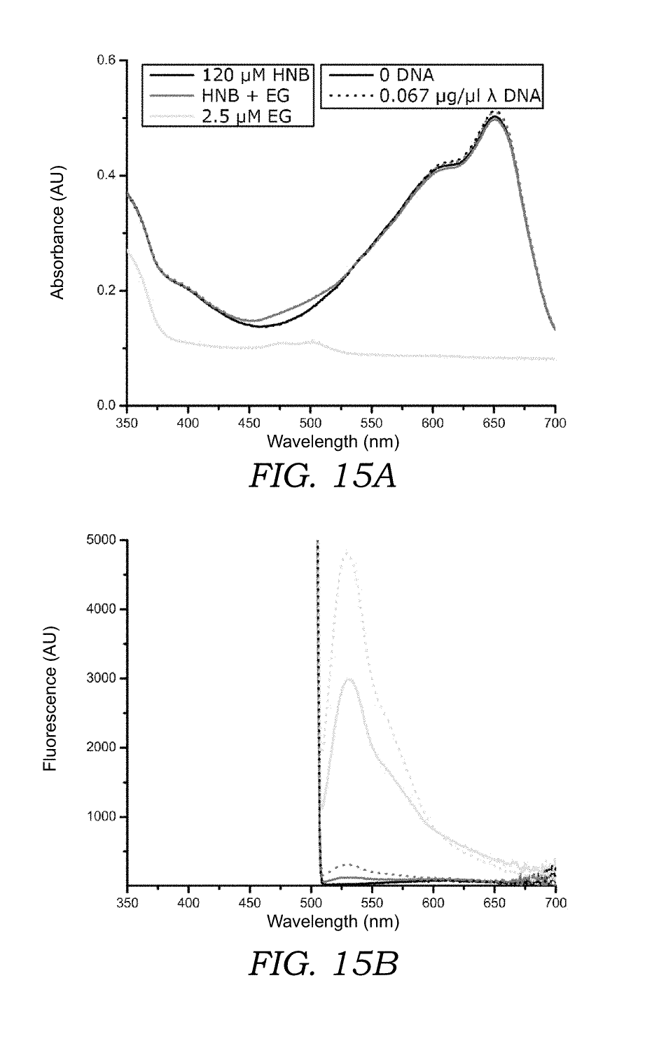

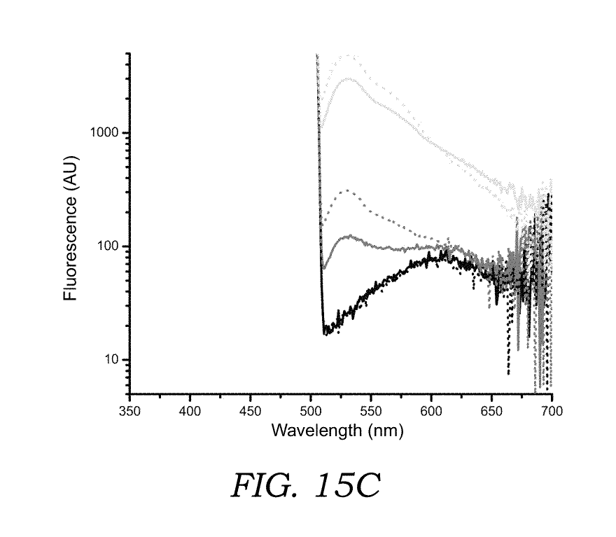

While SYBR® Green shows the greatest fluorescence change, giving the highest signal to background, previous studies have shown that SYBR® Green added prior to the amplification reaction greatly hinders the amplification process. The signal generated from the addition of DNA to acridine orange or EvaGreen® is not drastically higher than the background, and in some cases, cannot be distinguished from the background. The absorbance and emission curves for 2.5 μM EvaGreen® and 120 μM HNB as seen in FIGS. 15A-15C show that there is a significant amount of overlap between the wavelengths absorbed by HNB and the wavelengths emitted by EvaGreen® (between 525 nm to 575 nm). These absorption and emission spectra suggest that this affinity could also be accompanied by a FRET or quenching interaction. Comparing the emission spectra for the solution containing EvaGreen® versus EvaGreen® and HNB shows that the emission peak at approximately 535 nm is greatly reduced when HNB is present, and when DNA is added to the solution containing the dye combination, the emission at 535 nm increases significantly. This indicates that there is a shift in equilibrium for EvaGreen® to bind DNA compared to HNB. EvaGreen® association with DNA prevents the binding to HNB and also reduces the reduction of fluorescence due to resonance energy transfer or quenching with HNB for that molecule.

Next, examining the absorption and emission spectra of the LAMP reaction can show the role of the reaction buffer and DNA amplification. The reaction buffer contains a high concentration of magnesium, which is known to change the absorption spectra of HNB. However, the absorbance and emission spectra for EvaGreen®, HNB, the combination of the two in 8 mM magnesium, which is the concentration of the magnesium in the LAMP reaction solution, and the corresponding spectra in the LAMP reaction mixture have key differences. The absorbance spectra in the LAMP reaction is shifted towards higher wavelengths when compared to the magnesium buffered solution. Additionally, the emission spectra for the EvaGreen®, HNB, and dye combination have differing profiles in the LAMP reaction mixture versus the magnesium buffered solution.

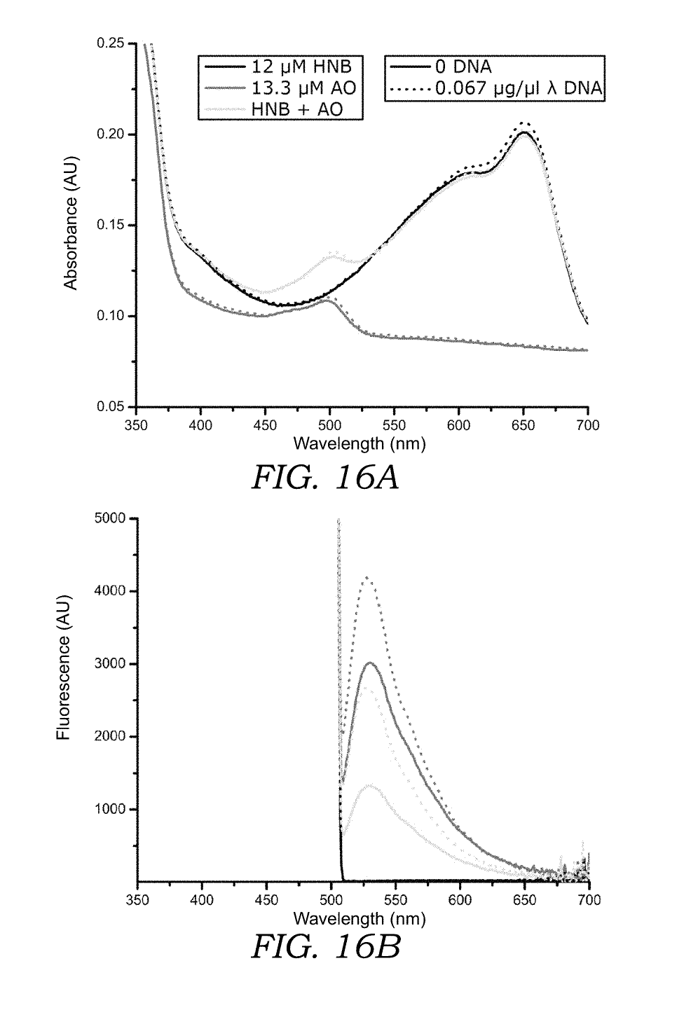

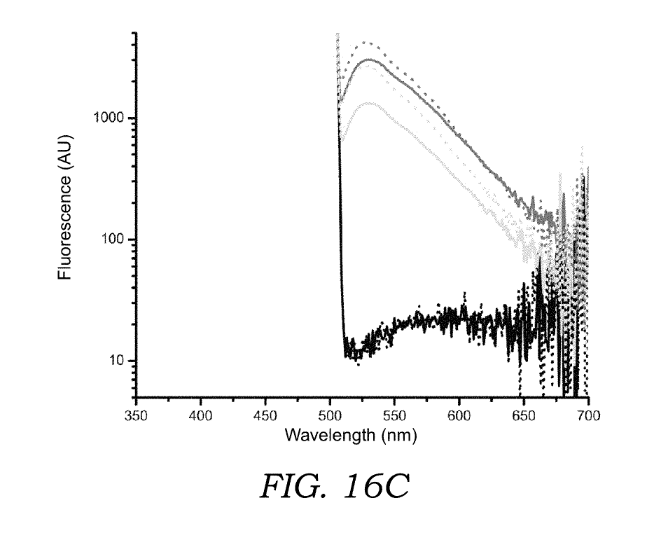

The emission spectrum for acridine orange is very similar in profile to EvaGreen®, with a single peak near 535 nm. FIG. 16A illustrates the absorption spectra for 13.3 μM acridine orange both with and without DNA. Also illustrated is the absorption spectra for 12 μM HNB (with and without DNA) and the absorption spectra of HNB and acridine orange (with and without DNA). FIG. 16B illustrates a linear plot of the emission spectra for 13.3 μM acridine orange with 12 μM of HNB. FIG. 16C illustrates a logarithmic plot of the emission spectra for 13.3 μM acridine orange with 12 μM HNB with and without DNA. Because EvaGreen® is a dimer of acridine orange, it follows that the emission spectra would be related. Like EvaGreen®, the emission at 535 nm is diminished by the addition of HNB to a solution containing acridine orange, and the significant increase in emission at 535 nm is able to be discerned when in the presence of DNA.

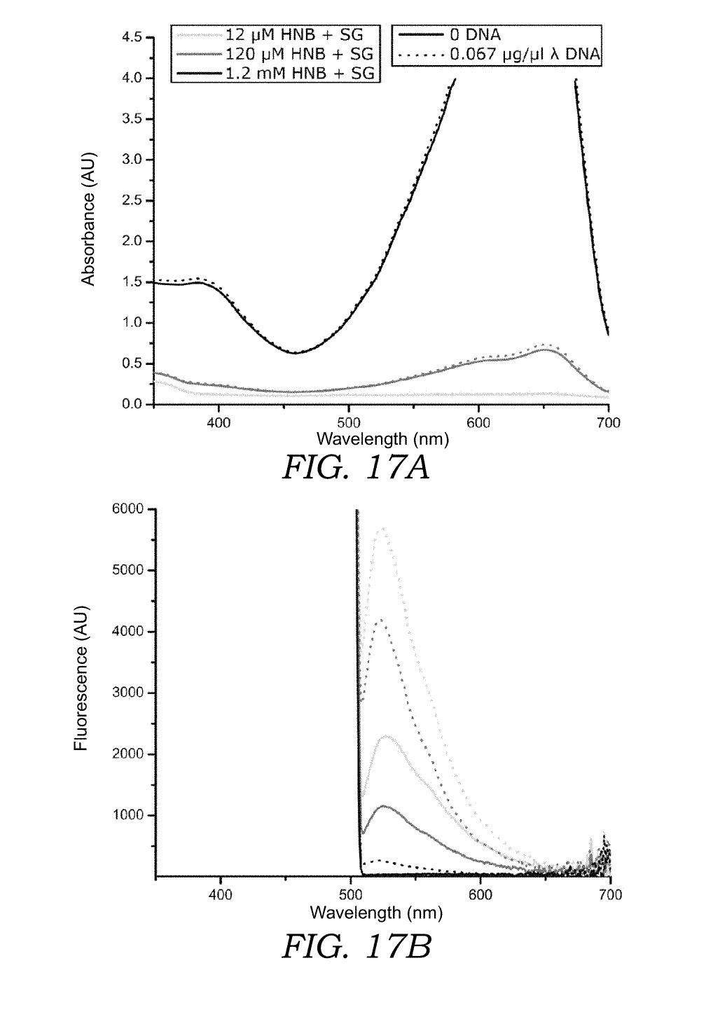

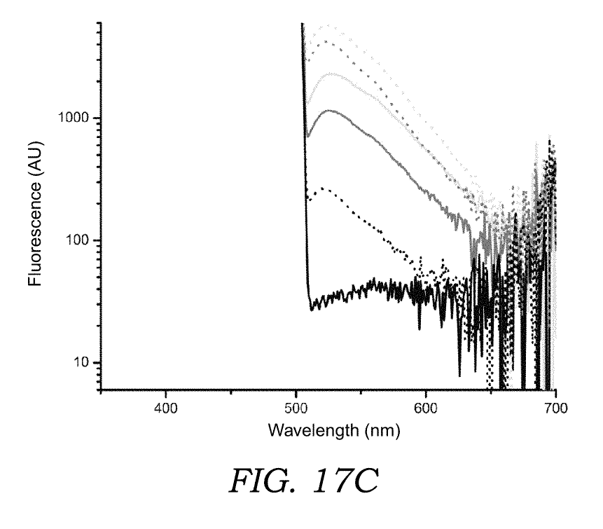

The lower affinity of SYBR® Green with HNB necessitates a larger concentration of HNB to effectively sequester this intercalator dye and prevent a high background fluorescence level, as shown in FIG. 17A-C. As shown in FIGS. 17B-C, higher concentrations of HNB might be more favorable for usage in amplification reactions, as higher HNB concentrations, for example 1.2 mM, result in a larger fluorescence emission fold change of SYBR® Green after the addition of DNA compared to lower concentrations.

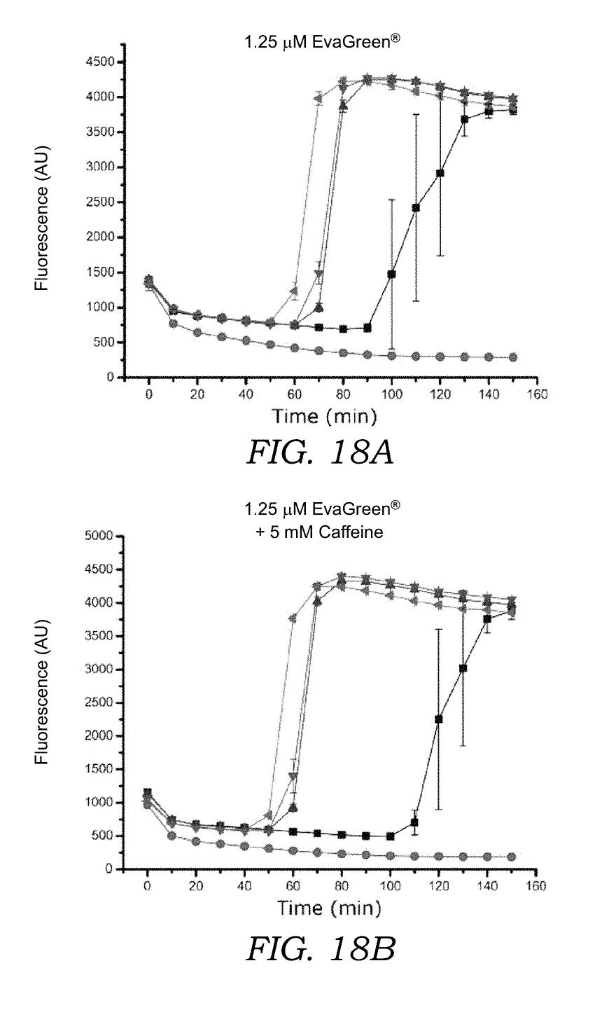

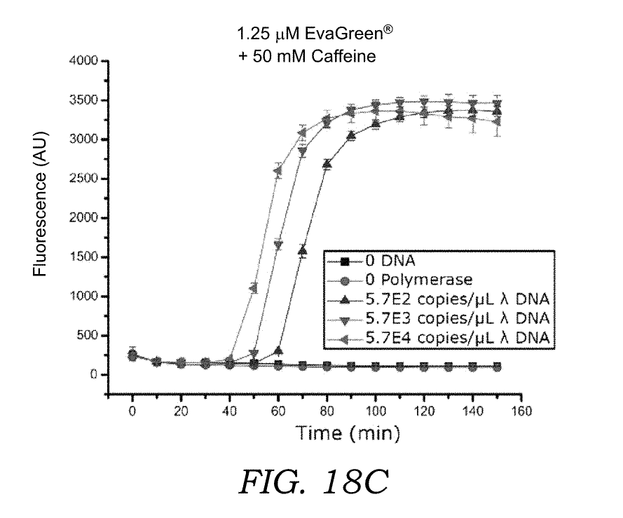

Finally, high concentrations of caffeine, which has a conjugated molecular structure that shares some similar characteristics with HNB, were added to EvaGreen® and demonstrated similar effects as the addition of HNB to EvaGreen®. FIGS. 18A-18C illustrate, respectively, real-time measurements of λ DNA amplification with LAMP with 1.25 μM EvaGreen® (no caffeine), 1.25 μM EvaGreen® and 5 mM caffeine, and 1.25 μM EvaGreen® and 50 mM caffeine. The addition of caffeine generated a more stable background fluorescence, visible in the 0 polymerase negative control with increased temperature for the length of the amplification reaction. Additionally, the fluorescence fold change increased from around 2.5 to 14 with the addition of 50 mM caffeine, which is a high level of caffeine. In the absorbance spectra for caffeine, however, there is minimal absorbance across all wavelengths. This finding suggests that the improvements to the fold change and fluorescence stability with increased temperatures by the addition of caffeine to EvaGreen® are not due to FRET interactions. Furthermore, the improvements in fold change between EvaGreen® and HNB are a result of a combination of FRET effects and binding/sequestering interactions that mitigate interference of intercalating dyes on amplification.

The interaction between EvaGreen® and HNB decreases the interactions between the intercalating dye and DNA that inhibit the DNA amplification reaction. Additionally, HNB sequesters and acts to quench the background fluorescence from EvaGreen® when not complexed with nucleic acids, increasing the fluorescence fold change over background upon nucleic acid amplification. Because the combination of both components do not interfere with the amplification process, the fluorescence can then be monitored with higher accuracy in real-time as the reaction proceeds and improved digital and portable readouts are possible with this improved readout system. Sequestering and quenching of intercalating dye fluorescence with negatively charged dyes that have aromatic ring structures is also possible.

In an embodiment separate to analyzing the nucleic acid products of amplification reactions, the dye mixture of intercalator and sequesterer can be used to directly readout the concentration of DNA in a solution by measuring fluorescence intensity of that solution or sample containing DNA. Because of the increased stability of the intercalator:sequesterer complex in solution, solutions can be stored and readout intensity will remain stable even in exposure to light and temperature fluctuations. Therefore, lengthy calibration of the intensity used known standards to identify a specific DNA concentration can be avoided. Varying ranges of the DNA solution concentration can be interrogated by changing the sequesterer concentration in the mixture. In this alternative embodiment, with reference to FIG. 1, the mixture would include the fluorescent intercalating dye 12, the quencher/sequestration agent 14, and the sample 14 which contains DNA therein. Optionally, a buffer solution may also be added to the mixture in this embodiment.

While embodiments of the present invention have been shown and described, various modifications may be made without departing from the scope of the present invention. The invention, therefore, should not be limited, except to the following claims, and their equivalents.

1. A fluorescent dye and quencher mixture for reporting on nucleic acid amplification from a sample comprising:
a fluorescent intercalating dye;
hydroxynapthol blue (HNB); and
primers, dNTPs, and a nucleic acid polymerizing enzyme or fragment thereof.

2. The mixture of claim 1, wherein the enzyme comprises polymerase or a fragment thereof.

3. The mixture of claim 1, wherein the enzyme comprises ligase or helicase and polymerase or fragments thereof.

4. (canceled)

5. The mixture of claim 1, wherein the fluorescent intercalating dye comprises a dimeric fluorescent dye having an emission peak at around 530 nm.

6. The mixture of claim 1, wherein the fluorescent intercalating dye comprises a cyanine dye having an emission peak at around 520 nm.

7. The mixture of claim 1, wherein the fluorescent intercalating dye comprises acridine orange.

8. A fluorescent dye and quencher mixture for reporting on nucleic acid amplification from a sample using loop-mediated isothermal amplification (LAMP) comprising:
a fluorescent intercalating dye;
hydroxynapthol blue (HNB); and
LAMP primers, dNTPs, LAMP reaction buffer, and DNA polymerase or a fragment thereof.

9. The mixture of claim 8, wherein the fluorescent intercalating dye comprises a dimeric fluorescent dye having an emission peak at around 530 nm.

10. The mixture of claim 8, wherein the fluorescent intercalating dye comprises a cyanine dye having an emission peak at around 520 nm.

11. The mixture of claim 8, wherein the fluorescent intercalating dye comprises acridine orange.

12. The mixture of claim 1, wherein the sample contains nucleic acid at concentration below 60 copies/μl.

13. The mixture of claim 1, wherein the fluorescent intercalating dye emits a fluorescent signal that is at least 20-50 fold above background following nucleic acid amplification of the sample.

14. The mixture of claim 9, wherein the concentration of the dimeric fluorescent dye is around 5 μM.

15. A fluorescent dye and quencher mixture for reporting on nucleic acid amplification from a sample comprising:
a fluorescent intercalating dye;
caffeine; and
primers, dNTPs, and a nucleic acid polymerizing enzyme or fragment thereof.

16. The mixture of claim 15, wherein the mixture contains at least 50 mM caffeine.

17. A method of using the mixture of claim 1, comprising:
forming a plurality of small volumes from the mixture;
imaging the plurality of small volumes; and
identifying a subset of the plurality of small volumes that emit a positive fluorescent signal.

18. The method of claim 17, wherein the positive fluorescent signal comprises a fluorescent signal that is at or above a pre-defined fluorescent intensity level.

19. The method of claim 17, further comprising counting the number of small volumes from the plurality that emit the positive fluorescent signal.

20. The method of claim 17, further comprising calculating a nucleic acid concentration in the sample based on the number of small volumes that emit a positive fluorescent signal.

21. The method of claim 17, wherein the small volumes comprise microwells, droplets, or emulsions.

22. (canceled)

23. A method of improving the fluorescent reporting of a nucleic amplification process that uses a fluorescent intercalating dye, the method comprising:
providing a sample containing a nucleic acid sequence to be amplified;
adding a mixture containing the fluorescent intercalating dye, hydroxynapthol blue (HNB), dNTPs, primers, and a nucleic acid polymerizing enzyme or fragment thereof.

24. The method of claim 23, wherein the fluorescent intercalating dye and HNB are present together at the beginning of the nucleic acid amplification process.

25. A fluorescent dye and quencher mixture for reporting on nucleic acid concentration from a sample comprising:
a sample containing deoxyribonucleic acid (DNA);
a fluorescent intercalating dye; and
hydroxynapthol blue (HNB).
